(19) United States
(12) Patent Application Publication
(10) Pub. No.: US 2015/0119648 A1
(43) Pub. Date: Apr. 30, 2015

(54) SELF-RETAINING RETRACTOR FOR HIP REPLACEMENT SURGERY

(71) Applicants: Steven L. Barnett, Villa Park, CA (US); Theodore Firestone, Scottsdale, AZ (US)

(72) Inventors: Steven L. Barnett, Villa Park, CA (US); Theodore Firestone, Scottsdale, AZ (US)

(21) Appl. No.: 14/064,043

(22) Filed: Oct. 25, 2013

(51) Int. Cl. A61B 17/02

(52) U.S. Cl. CPC A61B 17/0206
USPC 600/226

(57) ABSTRACT

The field of the invention relates to systems and methods for surgical retractor, and more particularly to systems and methods for self-retaining retractor used in hip replacement surgery. In an embodiment, a retractor for surgery comprises a pair of handles, a locking ratchet, a first arm comprising a first portion, a hinge joint, and a second portion having a modular blade system; and a second arm comprising a first portion, a universal locking joint, a locking lever, and a second portion having a generally triangular shaped end. In an embodiment, the modular blade system has an interchangeable blade. In another embodiment, the second portion of the second arm has a ball shaped end. In another embodiment, the second portion of the second arm has a female end. In another embodiment, a female broach system comprises a female broach having a generally triangular aperture at one end, an extraction pattern on one side, and compaction pattern on the remaining sides; a male acetabular self-retaining broach insert; and a male trunion trial.

### FIELD OF THE INVENTION

The field of the present invention generally relates to surgical devices, and more particularly to a self-retaining retractor used in hip replacement surgery.

### BACKGROUND OF THE INVENTION

Total hip replacement surgery requires surgical exposure of the hip joint in order to insert the prosthetic replacement. Generally, in a total hip replacement surgery, the surgeon makes an incision opening approximately 10-30 cm long in various locations depending on which defined approach is being used to expose the joint. The incision is stretched open to 7 to 8 cm wide by using multiple surgical retractors. One or more surgical assistants are needed to hold these retractors to keep the incision open during the operation. To expose the acetabulum (hip socket), mobilization and retraction of the femur is necessary for visualization. Typically this is undertaken by placing multiple angled retractors over the rim of the socket. This is done using retractors of various sizes and shapes based on the preference of the surgeons. The presence of assistants and retractors results in less room for the surgeon to operate, and increases the cost of the surgery. Some current self-retaining retractor systems still take up much space and are difficult and/or clumsy to use.

Further, total hip replacement surgery utilizing an anterior approach usually proceeds by exposing the joint and mobilizing the soft tissues so that the proximal femur can be displaced posteriorly for visualization and instrumentation of the socket. After the socket instrumentation is completed, the femur is prepared by broaching and insertion of the femoral stem. Socket preparation in this manner is often difficult because the femur can be difficult to retract and blood egress from the femoral canal typically drains in the field of view.

Accordingly, improved systems and methods for a self-retaining hip replacement surgery retractor that does not take up much space and improves visualization are desirable.

### SUMMARY OF THE INVENTION

The field of the invention relates to systems and methods for a surgical retractor, and more particularly to systems and methods for self-retaining retractor used in hip replacement surgery.

In an embodiment, a retractor for surgery comprises a pair of handles, a locking ratchet, a first arm comprising a first portion, a hinge joint, and a second portion having a modular blade system; and a second arm comprising a first portion, a universal locking joint, a locking lever, and a second portion having a generally triangular shaped end. In an embodiment, the modular blade system has an interchangeable blade. In another embodiment, the second portion of the second arm has a ball shaped end. In another embodiment, the second portion of the second arm has a female end. In another embodiment, a female broach system comprises a female broach having a generally triangular aperture at one end, an extraction pattern on one side, and compaction pattern on the remaining sides; a male acetabular self-retaining broach insert; and a male trunion trial.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims.

### BRIEF DESCRIPTION OF THE DRAWINGS

In order to better appreciate how the above-recited and other advantages and objects of the inventions are obtained, a more particular description of the embodiments briefly described above will be rendered by reference to specific embodiments thereof, which are illustrated in the accompanying drawings. It should be noted that the components in the figures are not necessarily to scale, emphasis instead being placed upon illustrating the principles of the invention. Moreover, in the figures, like reference numerals designate corresponding parts throughout the different views. However, like parts do not always have like reference numerals. Moreover, all illustrations are intended to convey concepts, where relative sizes, shapes and other detailed attributes may be illustrated schematically rather than literally or precisely.

FIG. 1 is a perspective view of a surgical retractor according to an embodiment of the invention.

FIG. 1A is a schematic view of a blade system according to an embodiment of the invention.

FIG. 2 is perspective view of a female broach system according to an embodiment of the invention.

FIG. 3 is another perspective view of a surgical retractor according to an embodiment of the invention.

FIG. 4 is another perspective view of a surgical retractor according to an embodiment of the invention.

FIG. 4A is a schematic view of a female end of a surgical retractor according to an embodiment of the invention.

FIG. 4B is a schematic view of a conventional broach for use with the surgical retractor of FIG. 4 of the invention.

FIG. 5 is a view of an intraoperative example of the surgical retractor of FIG. 3 and the female broach system being used in an anterior hip replacement surgery according to an embodiment of the invention.

FIG. 6 is a view of an intraoperative example of the surgical retractor of FIG. 1 and the female broach system being used in an anterior hip replacement surgery according to an embodiment of the invention.

FIG. 7 is a view of an intraoperative example of the surgical retractor of FIG. 4 and a conventional broach being used in an anterior hip replacement surgery according to an embodiment of the invention.

### DETAILED DESCRIPTION OF THE PREFERRED EMBODIMENTS

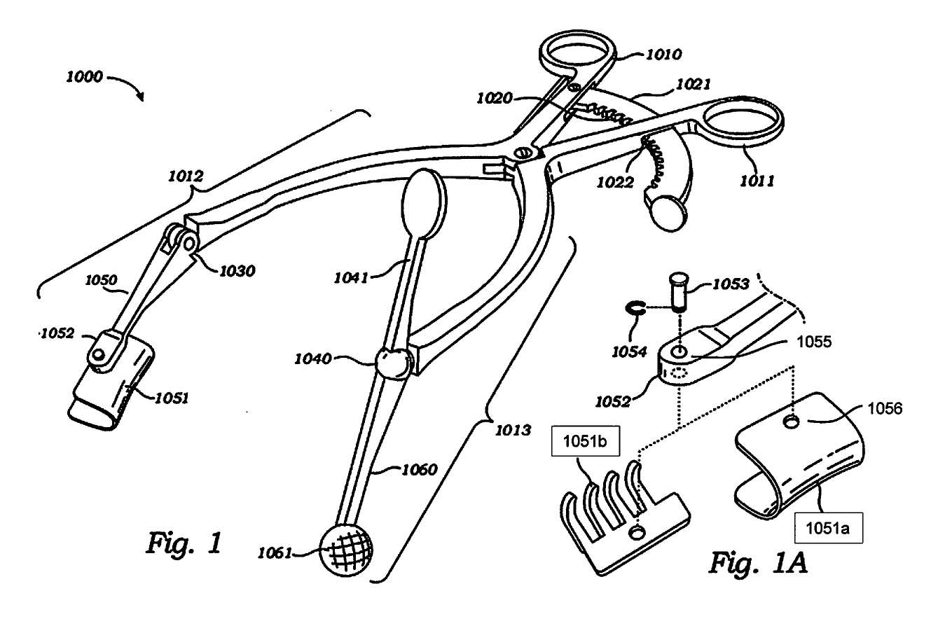

Turning to FIG. 1, according to an embodiment, an acetabular self-retaining retractor system 1000 is shown. The acetabular self-retaining retractor system 1000 generally comprises handles 1010, 1011 with finger rings, a locking ratchet 1020, an arm 1012 having a hinge joint 1030, an anterior capsule arm (or anterior retractor arm) 1050 and a modular blade system 1051, an arm 1013 having a universal locking joint 1040, with locking lever 1041, and a posterior retractor arm 1060 having a ball-tip 1061.

The acetabular self-retaining retractor system 1000 has an adjustable self-locking mechanism 1020. The self-locking mechanism 1020 includes a locking ratchet 1021 with a serrated (teeth) edge away from the finger rings of handles 1010, 1011. The locking ratchet 1021 has an end which is anchored in an elongated aperture on the inner side of the handle 1010, allowing the locking ratchet 1021 to pivot. The locking ratchet 1021 moves through an elongated aperture 1022 on the side of the handle 1011. As the handles 1010, 1011 are spread, the locking ratchet 1021 slides freely through the aperture 1022, but will lock in place when the spreading movement stops. In the locked position, the teeth on the locking ratchet 1021 prevent reversal movement of the locking ratchet 2021. The locking ratchet 1021 has an enlarged end, which serves as a stopper to prevent the ratchet from sliding off the handle 1022. In an embodiment, the locking ratchet 1021 is a conventional ratchet.

The arm 1012 comprises a hinge joint 1030, and an anterior capsule arm (or anterior retractor arm) 1050. The hinge joint 1030 allows the anterior capsule arm 1050 to rotate in one plane. The anterior capsule arm 1050 further comprises a modular blade system 1051 coupled to the end 1052. As shown in FIG. 1A, the modular blade system 1051 includes an interchangeable blade 1051a. The blade 1051a has a curve shape with an upper curve and a lower curve to secure the capsule effectively. The upper curve of the blade 1051a has a round opening 1056 sized to fit a pin 1053. The blade 1051a is coupled to the end 1052 of the anterior capsule arm 1050 using the pin 1053 and pin clip 1054. The pin 1053 is inserted through the round opening 1055 of the end 1052. As such, the blade 1051a pivots around the pin 1053. In an embodiment, the blade 1051a can have different sizes in length, width, or height. In another embodiment, a blade 1051b may be used with the modular blade system 1051. The blade 1051b has some serrations, forming one or more fingers. The blade's 1051 fingers have blunt, non-pointed ends.

According to an embodiment (not shown), the blade system 1051 has a blade 1051a which is permanently attached to the end 1052 of the anterior capsule arm 1050. In this embodiment, the blade 1051a may or may not pivot around the pin 1053.

The arm 1013 comprises a universal locking joint 1040 with locking lever 1041, and a posterior retractor arm 1060. The posterior retractor arm 1060 further comprises a ball shaped end 1061. When in an unlocked position, the universal locking joint 1040 allows for omnidirectional rotation of the posterior retractor arm 1060. The locking lever 1041 engages (locks) the universal locking joint 1040 to hold the posterior retractor arm 1060 in a fixed position. In an embodiment, the universal locking joint 1040 is a conventional locking joint.

As will be described in more detail below, the anterior capsule arm 1050 and posterior retractor arm 1060 are used to expose the acetabulum (hip socket). With the use of the self-locking mechanism 1020 and the universal locking joint 1040, the self-retaining retractor system 1000 can keep the anterior capsule arm 1050 and posterior retractor arm 1060 locked in place during the surgery, without the need for an assistant to hold the retractor.

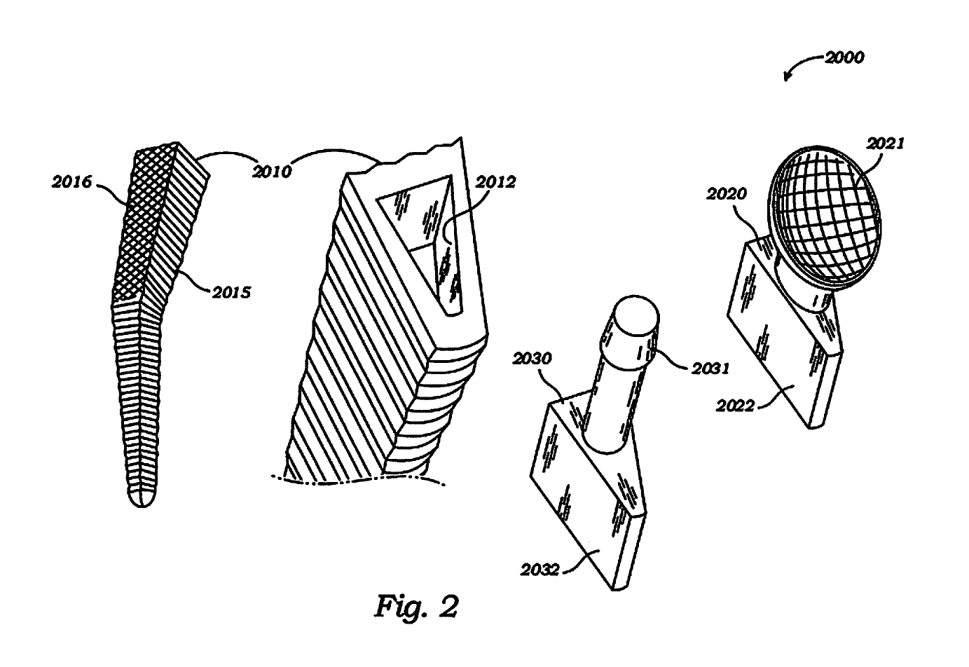

Turning to FIG. 2, a female broach system 2000 according to an embodiment is shown. Generally, the self-retaining retractor system 1000 articulates with the female broach system 2000 which has been previously placed into the femoral canal to facilitate exposure during the acetabular preparation step of total hip replacement surgery.

The female broach system 2000 comprises a female broach 2010, a male acetabular self-retaining broach insert 2020, and a male trunion trial 2030. Generally, a broach is used to shape and hollow out the femoral canal to the desired shape of a femoral stem which will be inserted into the femur. The female broach 2010 has a conventional compaction pattern 2015 on the anterior, posterior, and medial sides (when it is inserted into the femur). The compaction pattern 2015 compacts the spongy bone of the femur. The female broach 2010 has an extraction pattern 2016 on the lateral side (when it is inserted into the femur). The extraction pattern 2016 extracts the spongy bone of the femur as the female broach 2010 is removed from the femur. This helps insure that the broach will be placed into the femoral canal in optimal position (avoiding a varus malposition of the stem). The other remaining sides of the female broach 2010 also have compaction pattern. The female broach 2010 has a generally triangular aperture 2012 at the proximal broach end to receive the base 2022 of the male acetabular self-retaining broach insert 2020. As such, the generally triangular aperture 2012 has a depth that is generally equal to the height of the base 2022 of the male acetabular self-retaining broach insert 2020. The male acetabular self-retaining broach insert 2020 also comprises a cup-shaped head 2021 that articulates with the ball shaped end 1061 of the self-retaining retractor system 1000, as will be described in more detail below. The male trunion trial 2030 also comprises a base 2032 which is essentially similar to the base 2022 of the male acetabular self-retaining broach insert 2020, as the male trunion trial 2030 is also inserted into the female broach 2010, as will be described in more detail below. The male trunion trial 2030 also comprises a conventional neck 2031 to receive a conventional femoral head.

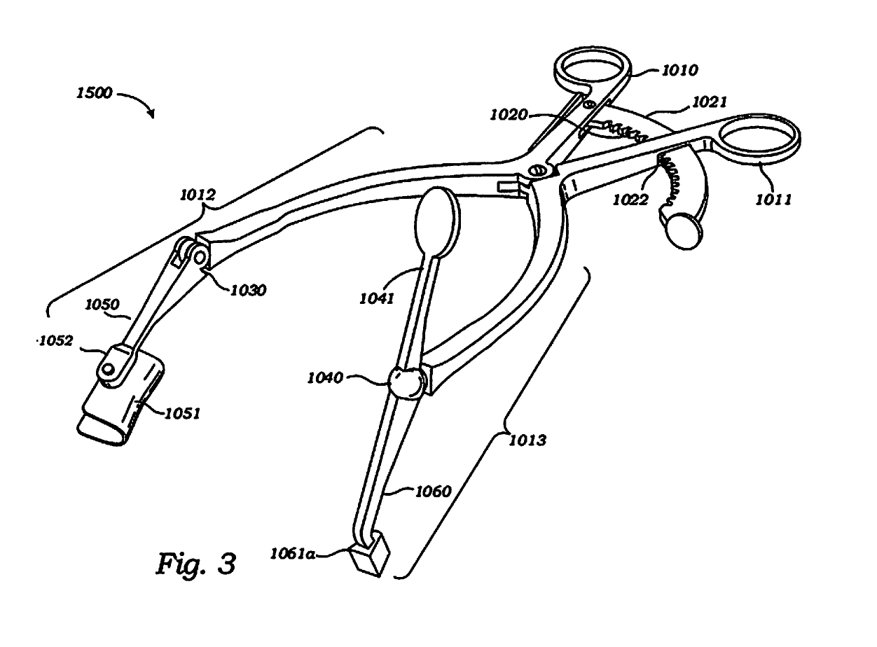

Turning to FIG. 3, according to an embodiment, the posterior retractor arm 1060 of the self-retaining retractor system 1500 has a generally triangular shaped end 1061a. The end 1061a is similar to the base 2022 of the broach insert 2020 and the base 2032 of the male trunion 2030 (FIG. 2), such that the end 1061a can fit into the generally triangular aperture 2012 of the female broach 2010.

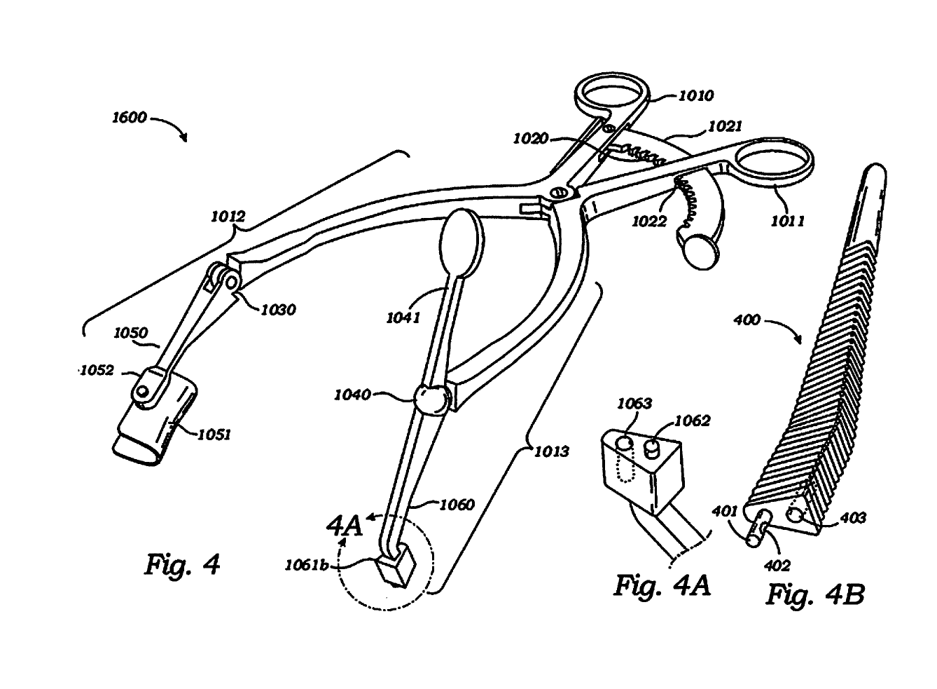

Turning to FIG. 4, according to an embodiment, the posterior retractor arm 1060 of the self-retaining retractor system 1600 has a female end 1061b. The female end 1061b, as shown in FIG. 4A, is used to articulate with a conventional male broach 400, as shown in FIG. 4B. The conventional male broach 400 has a stem (or finger) 401 at the proximal broach end. The stem 401 has an indentation (or recess) 402. The conventional male broach 400 also has a round recess (or bore) 403 at the proximal broach end. As such, to articulate with a conventional male broach 400, the female end 1061b of the self-retaining retractor system 1600 has a recess (or bore) 1063 to receive the stem 401, and a stem (or finger) 1062 sized to fit within the recess 403 of the conventional male broach 400. In an embodiment, the recess 1063 of the female end 1061b has a latch to engage the indentation 402 of the stem 401 of the conventional male broach 400. In an embodiment, the female end 1061b has a generally triangular shape. In another embodiment, the female end 1061b has a generally rectangular shape (not shown).

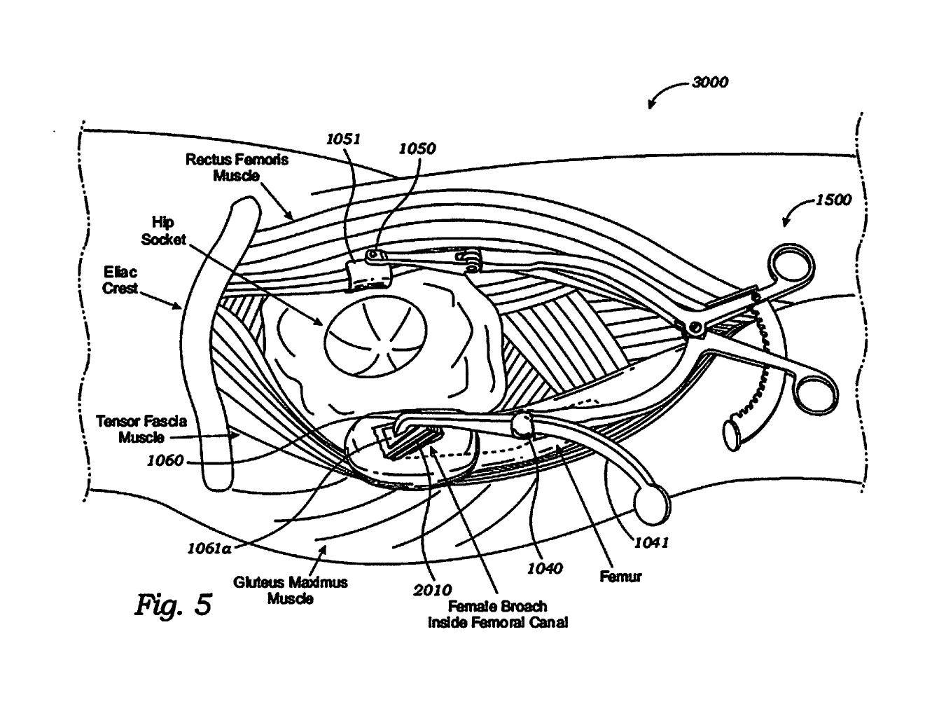

In a total hip replacement surgery the femoral bone is prepared for implantation by utilizing a series of gradually increasing broach sizes until adequate press-fit is achieved. Once this step is completed, the final femoral implant is placed until it seats firmly in the proximal femur. In a similar fashion, the acetabulum (socket) is prepared by exposing the area circumferentially and then preparing the bone with hemispherical reamers before inserting the hemispherical metal implant. Turning to FIG. 5, according to an embodiment, an intraoperative example 3000 of the acetabular self-retaining retractor system 1500 and the female broach system 2000 used in an anterior hip replacement surgery on a right hip is shown. Generally, when the self-retaining retractor system 1500 is used to keep the incision open to expose the acetabulum (hip socket), the anterior capsule arm 1050 retracts the rectus femoris muscle and anterior hip capsule with the modular blade system 1051. The posterior retractor arm 1060 engages the female broach 2010, which is placed in the femoral canal. This avoids potential damage to the acetabular rim as well as the proximal femur by eliminating any leverage exerted on these structures.

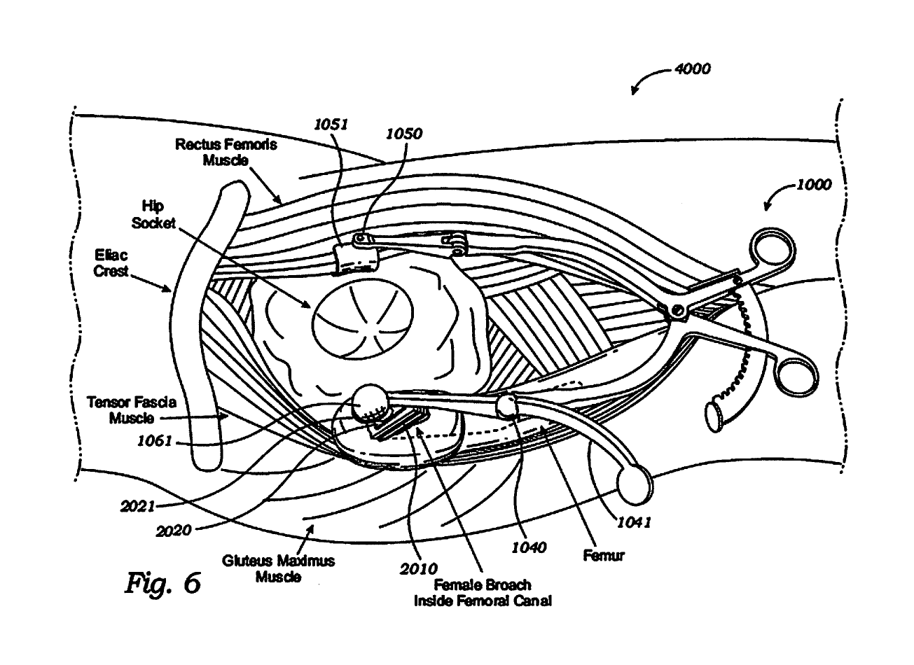

In an embodiment, where the posterior retractor arm 1060 has the generally triangular shaped end 1061a (self-retaining retractor system 1500 in FIG. 3), to engage the female broach 2010, the generally triangular shaped end 1061a is inserted into the generally triangular aperture 2012 of the female broach 2010. In another embodiment (as shown in FIG. 6), where the posterior retractor arm 1060 has the ball-tip 1061 end (self-retaining retractor system 1000 in FIG. 1), to engage the female broach 2010, the self-retaining broach insert 2020 is inserted into the generally triangular aperture 2012 of the female broach 2010, and the broach insert's 2020 cup-shaped head 2021 articulates with the ball shaped end 1061 (not seen) of the posterior retractor arm 1060. As the universal locking joint 1040 allows the posterior retractor arm 1060 to rotate substantially omnidirectionally, this facilitates the mobilization of the femur and the instrumentation of the femur and the acetabulum. Additionally, the problem of blood flow into the field is eliminated because the femoral canal is blocked by the inserted female broach system 2000.

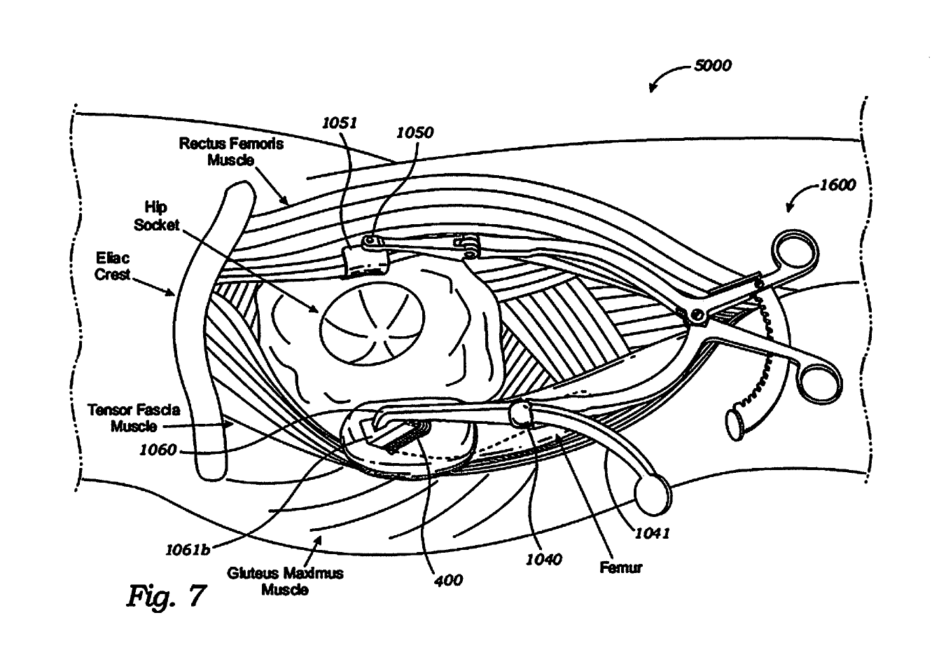

Turning to FIG. 7, according to an embodiment, an intraoperative example 5000 of the acetabular self-retaining retractor system 1600 having the female end 1061b (FIG. 4) used with a conventional male broach 400 in an anterior hip replacement surgery on a right hip is shown. To engage the conventional male broach 400, the female end 1061b of the posterior retractor arm 1060 articulates with the male broach 400 at the proximal broach end. The recess 1063 of the female end 1061b articulates with the stem 401 of the conventional male broach 400, the stem 1062 of the female end 1061b articulates with the recess 403 of the conventional male broach 400.

It is noted that using the self-retaining retractor systems 1000 or 1500 with the female broach system 2000, and using the self-retaining retractor system 1600 with a conventional broach 400 will eliminate the need for removal prior to instrumenting the acetabulum.

In the foregoing specification, the invention has been described with reference to specific embodiments thereof. It will, however, be evident that various modifications and changes may be made thereto without departing from the broader spirit and scope of the invention. For example, the reader is to understand that the specific ordering and combination of process actions described herein is merely illustrative, and the invention may appropriately be performed using different or additional process actions, or a different combination or ordering of process actions. For example, this invention is particularly suited for anterior hip replacement; however, the invention can be used for any (e.g., anterior, lateral, posterior) hip replacement in general. Additionally and obviously, features may be added or subtracted as desired. Accordingly, the invention is not to be restricted except in light of the attached claims and their equivalents.

1. A retractor for surgery comprising:
a first handle and a second handle;
a locking ratchet coupled to the first handle;
a first arm extended from the first handle comprising a first portion, a hinge joint, and a second portion having a modular blade system; and
a second arm extended from the second handle comprising a first portion, a universal locking joint, a locking lever, and a second portion having a generally triangular shaped end.

2. The retractor of claim 1, wherein the universal locking joint allows the second portion of the second arm to rotate in a substantial omnidirection.

3. The retractor of claim 1, wherein the locking lever locks the second portion of the second arm in a fixed position.

4. The retractor of claim 1, wherein the locking ratchet locks the handles in a fixed position.

5. The retractor of claim 1, wherein the modular blade system comprises an interchangeable blade which is coupled to the second portion of the first arm.

6. The retractor of claim 5, wherein the interchangeable blade has an upper curve and a lower curve.

7. A retractor for surgery comprising:
a first handle and a second handle;
a locking ratchet coupled to the first handle;
a first arm extended from the first handle comprising a first portion, a hinge joint, and a second portion having a modular blade system; and
a second arm extended from the second handle comprising a first portion, a universal locking joint, a locking lever, and a second portion having a ball shaped end.

8. The retractor of claim 7, wherein the locking lever locks the second portion of the second arm in a fixed position.

9. The retractor of claim 7, wherein the locking ratchet locks the handles in a fixed position.

10. The retractor of claim 6, wherein the blade has a serrated edge.

11-15. (canceled)

16. A retractor for surgery comprising:
a first handle and a second handle;
a locking ratchet coupled to the first handle;
a first arm extended from the first handle comprising a first portion, a hinge joint, and a second portion having a modular blade system; and
a second arm extended from the second handle comprising a first portion, a universal locking joint, a locking lever, and a second portion having a female end.

17. The retractor of claim 16, wherein the universal locking joint allows the second portion of the second arm to rotate in a substantial omnidirection.

18. The retractor of claim 16, wherein the locking lever locks the second portion of the second arm in a fixed position.

19. The retractor of claim 16, wherein the locking ratchet locks the handles in a fixed position.

20. The retractor of claim 16, wherein the female end of the second portion of the second arm articulates with a conventional male broach.

21. The retractor of claim 16, wherein the modular blade system comprises an interchangeable blade which is coupled to the second portion of the first arm.
